(19) United States
(12) Patent Application Publication
(10) Pub. No.: US 2020/0372982 A1
(43) Pub. Date: Nov. 26, 2020

(54) IMPUTING AN OUTCOME ATTRIBUTE TO A PERS RECORD MISSING AN OUTCOME ATTRIBUTE USING A STRUCTURED SITUATION STRING OR UNSTRUCTURED CASE NOTE TEXT ASSOCIATED WITH THE RECORD

(71) Applicant: KONINKLIJKE PHILIPS N.V., EINDHOVEN (NL)

(72) Inventors: Mariana NIKOLOVA-SIMONS, Eindhoven (NL); Jorn OP DEN BUIJS, Eindhoven (NL); Linda SCHERTZER, Framingham, MA (US); Debra ALPERT, Framingham, MA (US)

(21) Appl. No.: 16/767,619

(22) PCT Filed: Nov. 30, 2018
(86) PCT No.: PCT/EP2018/083070

§ 371 Date: May 28, 2020

Related U.S. Application Data
(60) Provisional application No. 62/594,215, filed on Dec. 4, 2017.

(51) Int. Cl. G16H 10/60; G16H 50/70; G16H 50/30; G06Q 50/26; H04M 3/537

(52) U.S. Cl. CPC G16H 10/60; G16H 50/70; H04M 3/537; G06Q 50/265; G16H 50/30

(57) ABSTRACT

A computing system (204) includes a memory device (208) configured to store missing outcome instructions (218) and a processor (206) configured to execute the missing outcome instructions. The instructions cause the processor to: evaluate records stored on a storage device (114) of a call center (106) of a system (102), wherein the storage device is configured to store electronic records of calls from a user site (104) to the call center, and each record includes at least a situation attribute field and an outcome attribute field, identify records of the stored records which include, at least one of, a value in the situation attribute or case note text in a case note database, but no value in the outcome attribute, and impute an outcome to the outcome attribute field of each identified record using at least one of the situation attribute and the case note text to predict the missing outcome.

### FIELD OF THE INVENTION

The following generally relates to imputing an outcome attribute to a Personal Emergency Response Service (PERS) electronic record missing an outcome attribute using a structured situation string or unstructured case note text associated with the record.

### BACKGROUND OF THE INVENTION

FIG. 1 schematically illustrates an example personal emergency response system (PERS) 102. In this example, the PERS 102 includes at least a user site 104 and a call center 106. The user site 104 includes a portable device 108 and a user site communication (USCOMM) system 110, and the call center 106 includes a call center communication (CCOMM) system 112 and a storage device 114. The user site and call center communication systems 110 and 112 are configured to communicate with each other via a telephone line, a cellular network, etc.

The portable device 108 is configured to be carried by a user (i.e. a subscriber of the PERS 102), e.g., as part of a pendent of a neckless, supported on a wristband or belt, etc. The portable device 108 includes at least a wireless transmitter, which is activatable via actuation of a push button, voice detection, etc. The portable device 108 may also include a microphone and a speaker. The transmitter, when activated, wirelessly transmits a signal to the user site communication system 110, which causes the user site communication system 110 to “call” the call center communication system 112. It is understood that USCOMM 110 may be located within device 108 or separate from it.

Personnel at the call center 106 assesses the call. Where the call is intentional and the user requires assistance, the personnel at the call center 106 may contact an informal responder (e.g. a neighbor, a family member, etc.), an emergency responder (an ambulance service, the police department, the fire department, etc.), etc. Information about each call is stored in a data structure/record 116 (e.g., as an electronic file in the storage device 114) that includes attribute fields for identifying at least a case identification, a type of the call, a situation/reason for the call, and an outcome of the call.

Jorn op den Buijs et al., “Predictive Modeling of Emergency Hospital Transport using Medical Alert Pattern Data: Retrospective Cohort Study,” iproc 2015; 1(1):e19, DOI: 10.2196/iproc.4772, indicates that this information has been used with predictive models to predict whether a user of the portable device 108 is at risk for emergency transport to a healthcare facility, and, if so, to notify a clinician of the user. In FIG. 1, the clinician is at a clinical site 118 (e.g., an office, a hospital, a current location of the clinician, etc.) that includes a clinical site communication (CSOMM) system 120. Likewise, the communication with the clinical site communication system 120 can be through a telephone line, a cellular network, etc.

The predictive models are trained and tested with the outcome attribute of records. However, the attribute fields in each record for each call from the user site 104 to the call center 106 are not always populated, and records without an outcome attribute cannot be used in training and/or testing of the predictive models because they do not include outcomes. Unfortunately, this may negatively impact the ability of the predictive models to accurately predict users at risk of emergency transport, and this may lead to less than optimal care and increased cost.

### SUMMARY OF THE INVENTION

Aspects described herein address the above-referenced problems and others.

In one aspect, a computing system includes a memory device configured to store missing outcome instructions and a processor configured to execute the missing outcome instructions. The instructions cause the processor to: evaluate records stored on a storage device of a call center of a system, wherein the storage device is configured to store electronic records of calls from a user site to the call center, and each record includes at least a situation attribute field and an outcome attribute field, identify records of the stored records which include, at least one of, a value in the situation attribute or case note text in a case note database, but no value in the outcome attribute, and impute an outcome to the outcome attribute field of each identified record using at least one of the situation attribute and the case note text to predict the missing outcome.

In another aspect, a method includes identifying stored records, which include at least a situation attribute field and an outcome attribute field, that have at least one of a situation attribute and case note text in a case note database, and no outcome attribute, and determining whether the situation attribute field includes a structured situation string or a reference to a case note in response to the situation attribute field including a situation attribute. The method further includes performing a structured situation string analysis to impute an outcome to one or more of the records missing an outcome attribute in response to the situation attribute field including the structured situation string, and performing a case note analysis to impute an outcome to one or more of the records missing an outcome attribute in response to at least one of the situation attribute field including the reference to the case note and the case note database including the case note text. The method further includes employing records with outcome attributes and imputed outcome attributes to generate predictive models, and employing the predictive models to predict a health state of a user.

In another aspect, a computer readable storage medium is encoded with computer readable instructions. The computer readable instructions, when executed by a processer, cause the processor to: identify stored records, which include at least a situation attribute field and an outcome attribute field, that have at least one of a situation attribute and case note text in a case note database, and no outcome attribute, determine whether the situation attribute field includes a structured situation string or a reference to a case note in response to the situation attribute field including a situation attribute, perform a structured situation string analysis to impute an outcome to one or more of the records missing an outcome attribute in response to the situation attribute field including the structured situation string, perform a case note analysis to impute an outcome to one or more of the records missing an outcome attribute in response to at least one of the situation attribute field including the reference to the case note and the case note database including the case note text, employ records with outcome attributes and imputed outcome attributes to generate predictive models, and employ the predictive models to predict a health state of a user.

The invention may take form in various components and arrangements of components, and in various steps and arrangements of steps. The drawings are only for purposes of illustrating the embodiments and are not to be construed as limiting the invention.

### BRIEF DESCRIPTION OF THE DRAWINGS

FIG. 1 schematically illustrates an example of a personal emergency response service (PERS).

FIG. 2 schematically illustrates an example system including the PERS and a computing system for at least imputing an outcome to an outcome field of a record missing an outcome attribute.

FIG. 3 illustrates non-limiting examples of the type attribute of a PERS record.

FIG. 4 illustrates non-limiting examples of the situation attribute of the PERS record, when the type attribute is incident.

FIG. 5 illustrates non-limiting examples of the outcome attribute of the PERS record, when the type attribute is incident.

FIG. 6 illustrates an example method for imputing an outcome to the outcome field of a PERS record.

FIG. 7 illustrates an example look-up table used with structured situation string analysis to impute an outcome.

FIG. 8 illustrates an example algorithm for identifying records with a situation attribute or a case note and no outcome attribute.

FIG. 9 illustrates an example algorithm for predicting values for missing outcomes based on case note analysis.

FIG. 10 illustrates an example algorithm for blending predicted values to produce a single imputed outcome.

FIG. 11 illustrates a non-limiting example of blending rules for two predicted values.

FIG. 12 illustrates a non-limiting example algorithm for confirming an imputed outcome.

FIG. 13 illustrates a non-limiting example of a confirmation script for confirming an imputed outcome.

FIG. 14 illustrates example results after evaluating records for outcome and situation attributes for a particular type attribute.

FIGS. 15A and 15B illustrate example results after a structured situation string analysis at step 606 of FIG. 6.

FIG. 16 illustrates a summary of the structured situation string analysis results in FIGS. 15A and 15B.

FIG. 17 illustrates example results of step 902 of FIG. 9 showing prevalence of case outcome levels at cases with known outcome and case notes.

FIG. 18 illustrates performance of a model for predicting a first parameter of a missing outcome based on case notes analysis and step 910 in FIG. 9.

FIG. 19 illustrates performance of a model for predicting a second parameter of a missing outcome based on case notes analysis and step 910 in FIG. 9.

FIG. 20 illustrates examples blending rules for step 612 of FIG. 6 with the values P1=EMER ASSIST and P2=ER TRANSPORT.

FIG. 21 illustrates comparative results with and without imputation of an outcome attribute.

FIG. 22 illustrates bar graphs of positive predictive values for different cut-offs with and without imputation of an outcome attribute.

FIG. 23 illustrates bar graphs of sensitivities for different cut-offs with and without imputation of an outcome attribute.

FIG. 24 illustrates a summary of results for the type attribute incident showing the distribution of outcome levels, including those with partly missing outcomes (“EMER ASSIST-NO STATUS”).

FIG. 25 illustrates an example table that maps record case identification to case note.

### DETAILED DESCRIPTION OF EMBODIMENTS

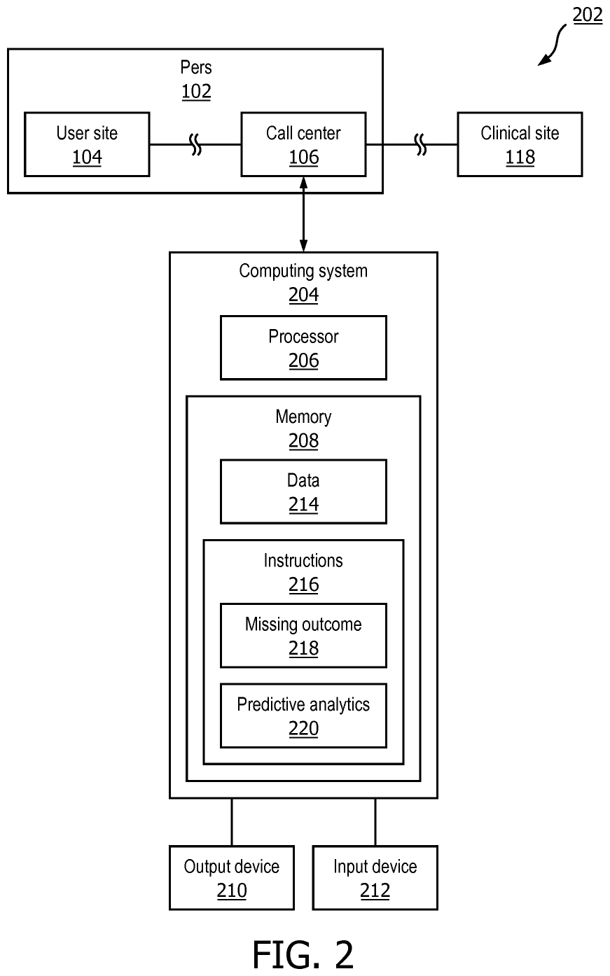

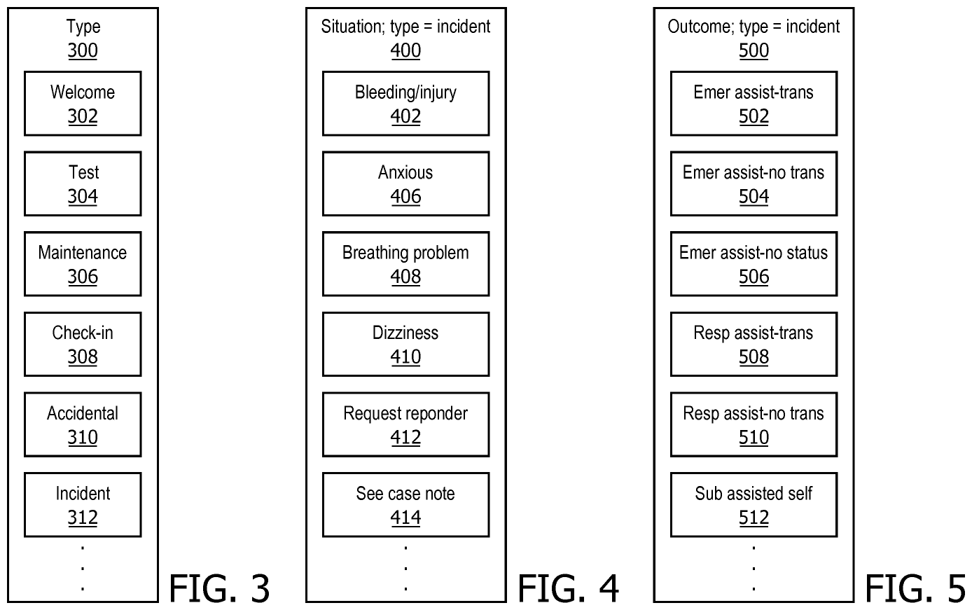

FIG. 2 schematically illustrates an example system 202, which includes the PERS 102, the clinical site 118, and a computing system 204. As discussed herein, the personnel at the call center 106 assesses a call from the user site 104 and records information about the call in a record (i.e. a data structure), which includes attribute fields for a case identification, a type, a situation, and an outcome of the call. FIGS. 3, 4 and 5 respectively show examples of type attributes 300, situation attributes 400 for the type attribute and outcome attributes 500 for the type attribute. It is to be understood that the illustrated example attributes 300, 400 and/or 500 are for explanatory purposes and are not limiting.

The type attribute 300 identifies a type of a call, which can be welcome 302, test 304, maintenance 306, check-in 308, accidental 310, incident 312, etc. The situation attribute 400 either identifies a reason for the call via a structured situation string or specifies that there is a case note with unstructured narrative text for the call. FIG. 4 shows example situation attributes 400 for the type incident and includes bleeding/injury 402, anxious 406, breathing problem 408, dizziness 410, request responder 412, and see case note 414. FIG. 5 shows example outcome attributes 500 for the type incident and includes emergency assist-transported (EMER ASSIST-TRANS) 502, emergency assist-not transported (EMER ASSIST-NO TRANS) 504, emergency assist-no status (EMER ASSIST-NO STATUS) 506, responder assist-transported (RESP ASSIST-TRANS) 508, responder assist-not transported (RESP ASSIST-NO TRANS) 510, and self-assistance (SUB ASSISTED SELF) 512.

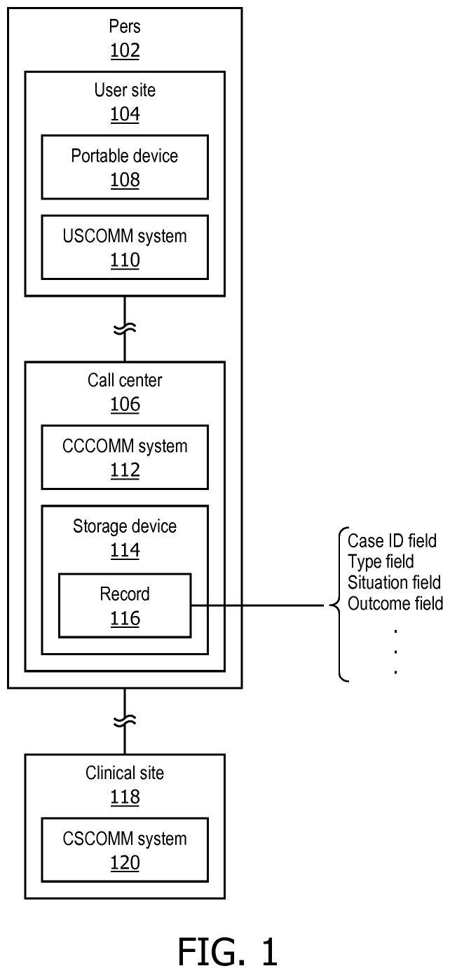

Returning to FIG. 2, the computing system 204 includes a hardware processor 206 (e.g., a central processing unit (CPU), a microprocessor, or the like). The computing system 204 further includes a computer readable storage medium (“memory”) 208 (which excludes transitory medium) such as physical memory and/or other non-transitory memory. The computing system 204 further includes an output device(s) 210 such as a display monitor, a speaker, etc., an input device(s) 212 such as a mouse, a keyboard, a microphone, etc. The computing system 204 is in communication with the storage device 114 (FIG. 1) at the call center 106. The computing system 204 may include the storage device 114 or be separate therefrom.

The memory 208 stores data 214, such as records, and computer readable instructions 216. The processor 206 is configured to execute the computer readable instructions 216. The illustrated computer readable instructions 216 include a missing outcome instruction set or algorithm 218 and a predictive analytics instruction set or algorithm 220. As described in greater detail below, the missing outcome instruction set or algorithm 218 includes instructions, which, when executed by the processor 206, cause the processor 206 to at least identify records with a situation attribute or an associated case note but no outcome attribute, and imputes an outcome attribute to the outcome field of the identified record using either the situation attribute or the case note if available.

The predictive analytics instruction set or algorithm 220 includes instructions that cause the processor 206 to predict users at risk of emergency transport based on predictive models, which are trained and tested on the outcomes in the records, and to generate notification, when needed, indicating a user is at risk for emergency transport with a specified time period (e.g., 30 days). An example product that performs such an analysis is the CareSage predictive analytics engine, a product of Koninklijke Philips N.V., a company headquartered in the NL. By also using imputed outcomes, the predictive analytics instruction set or algorithm 220 uses additional information that was not previously available, which can reduce patient risk, lower costs and/or improve patient outcomes.

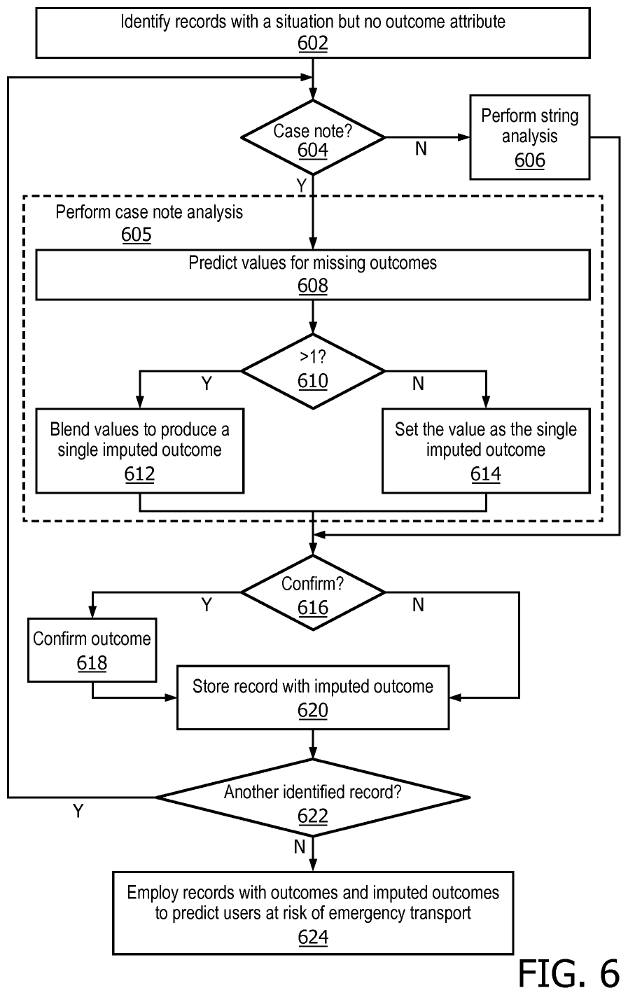

FIG. 6 illustrates an example of the missing outcome instruction set or algorithm 218.

At 602, stored call records are processed to identify, at least, records with a situation attribute (e.g., a string, a reference to a case note, and/or an associated case note text) but no outcome attribute. Act 602 is omitted where such records have already been identified. A more detailed example is provided below.

At 604, it is determined whether each of the identified records includes a structured situation string or a reference to a case note, and whether a case note database (e.g., in the storage device 114) has a case note corresponding to each record. For sake of brevity and explanatory purposes, reference to the term “case note” herein refers to: 1) the case note referenced in the situation; 2) the case note in the case note database; or 3) both the case note referenced in the situation attribute and the case note the case note database.

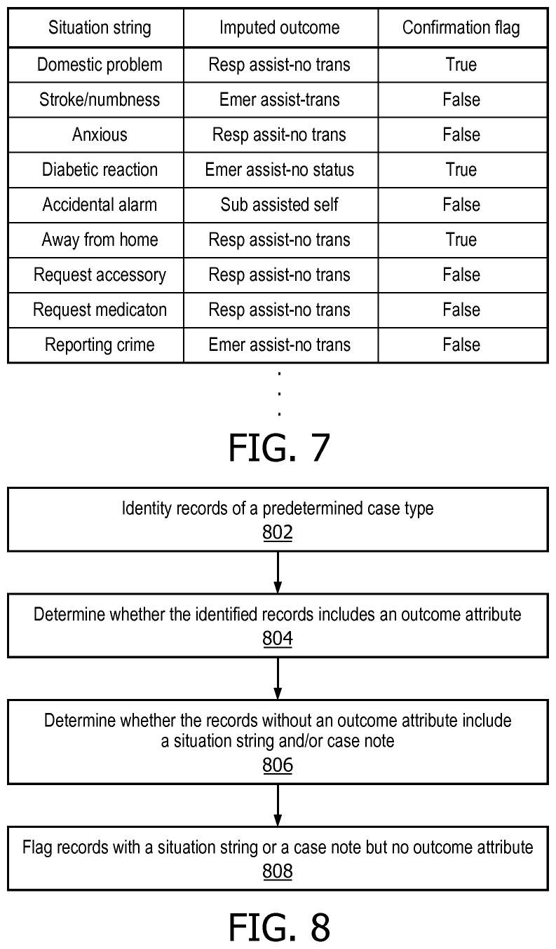

Returning to FIG. 6, for files with a structured situation string and no case note, at 606, a situation analysis is performed to impute a value for the outcome attribute based on the structured situation string. For example, the structured situation string can be mapped to an outcome using a predetermined look-up-table (LUT) that maps structured situation strings to outcomes. FIG. 7 shows an example of such a LUT. For instance, for a structured situation string of “ANXIOUS,” the imputed outcome is “RESP ASSIST-NO TRANS.”

Returning to FIG. 6, for records with a case note, at 605, a case note analysis is performed to impute a value for the outcome attribute based on the structured situation string. In this example, act 605 includes acts 608, 610, 612 and 614.

At 608, one or more new predictive models are employed to predict the value for the missing outcome attribute based on the case notes. A more detailed example is provided below.

At 610, it is determined whether there is a single predictive model or multiple predictive models utilized to impute the missing outcome attribute.

If there is more than one predicted value, then at 612, the predicted values are combined or blended according to predetermined assessment rules to produce a single imputed outcome for the file. A more detailed example is provided below.

If there is only one predictive model, at 614, the model output value is the single imputed outcome for the record.

At 616, it is determined whether the imputed outcome (from act 606, 612 or 614) is to be confirmed. For example, this information can be in the LUT of FIG. 7, which shows confirmation for “DOMESTIC PROBLEM”/“RESP ASSIST-NO TRANS” (i.e. CONFIRMATION FLAG=TRUE), and no confirmation for “ANXIOUS”/“RESP ASSIST-NO TRANS” (i.e. CONFIRMATION FLAG=FALSE).

Returning to FIG. 6, if the imputed outcome is to be confirmed, then at 618, the imputed outcome is confirmed. This can be achieved, either by retrieving a call script template and initiating an automated call to either a user or a responder to the user (a more detailed example is provided below), or by a performing an EHR analysis if EHR data are available.

If the imputed value is not to be confirmed or after confirmation, at 620, the record is populated with the single imputed outcome and stored.

At 622, it is determined whether there is another identified record.

If there is another record, then acts 604-622 are repeated for the record.

If there is no other identified record to process, at 624, the records with imputed outcomes can be stored, further processed, etc. For example, in one non-limiting instance, the records with imputed outcomes can be used along with the records with outcomes with predictive analytics to predict a health state of a user, e.g., users at risk of emergency transport and notify the clinical site 118, if needed, that a user(s) is at risk for emergency transport based on the predicted outcome.

FIG. 8 illustrates an example algorithm for act 602 of FIG. 6 to identify records with a situation attribute and no outcome attribute.

At 802, the stored records are processed to identify files of a predetermined case type (e.g., incident). For example, the computing system 204 receives a user input, via the input device 212, which indicates a case type of interest, and the processor 206 reads the value of the type attribute of each file and identifies the files that include a type attribute that is equal to the case type of interest—the predetermined case type.

At 804, each identified record is processed to determine whether the record includes an outcome attribute. For example, the processor 206 reads the value of the outcome attribute of a record and identifies whether the record include an outcome attribute. This is repeated for the other identified records.

At 806, each of the records with no outcome attribute is processed to determine whether the record includes a structured situation string or a case note. For example, the processor 206 reads the value of the situation attribute of a record and searches the case note database, and identifies whether the records include a structured situation string or a case note. For the later, the processor 206 extracts the case identification from the record and searches the case note database (e.g., a table thereof) for the case identification. If the case identification is found in the case note database, the processor retrieves the corresponding case note text. FIG. 25 shows an example table 2500 that maps record case identification (ID) to case note text. This is repeated for the other identified records.

Returning to FIG. 8, at 808, records with at least one of a structured situation string or a case note and no outcome attribute are flagged as records for further processing to impute an outcome to the outcome attribute. This can be achieved via a software and/or other flag.

In one instance, records that include an outcome attribute are flagged as records that can be used for predictive analytics. Additionally, or alternatively, records that are missing an outcome attribute, a structured situation string and a case note are flagged as records that cannot be used for predictive analytics.

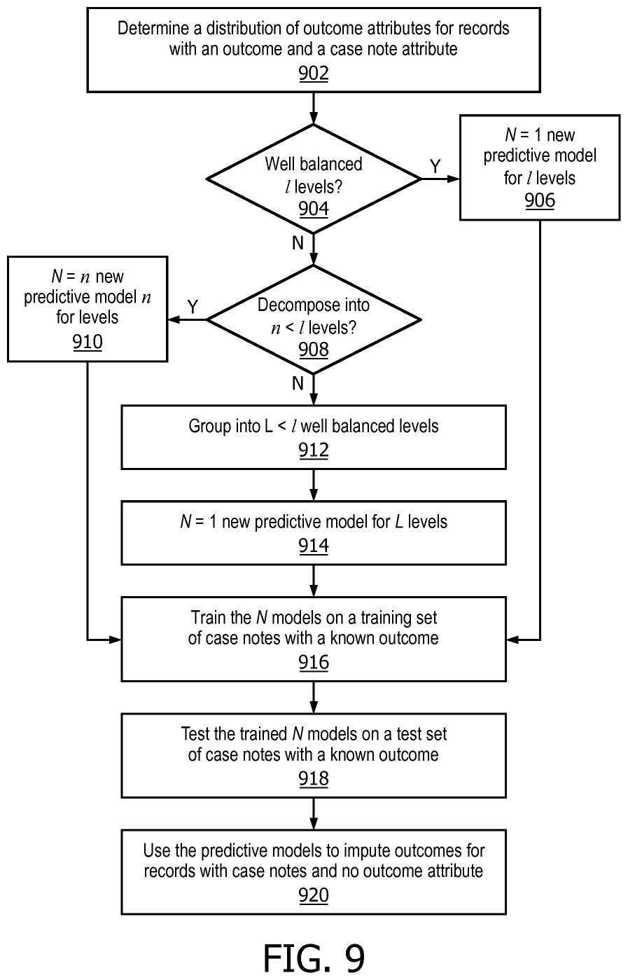

FIG. 9 illustrates an example algorithm for act 608 of FIG. 6 to predict values for missing outcomes, based on case notes.

At 902, records with an outcome attribute and a case note are processed to determine a distribution of l different outcomes l levels of the categorical variable. For example, using the six (6) outcome possibilities (i.e. l=6) from FIG. 5, the distribution will indicate a number of EMER ASSIST-TRANS outcomes, a number of EMER ASSIST-NO TRANS outcomes, a number of EMER ASSIST-NO STATUS outcomes, a number of RESP ASSIST-TRANS outcomes, a number of RESP ASSIST-NO TRANS outcomes, and a number of SUB ASSISTED SELF outcomes.

At 904, it is determined whether the distribution of the l levels is well balanced. In one instance, a well-balanced distribution is one in which a difference between a greatest occurrence of an outcome and a least occurrence of an outcome satisfies a predetermined threshold. For example, where there are at least two levels (l≥2), the greatest occurrence is Y for the EMER ASSIST-TRANS level, the least occurrence is Z for of SUB-ASSISTED SELF level, and the predetermined threshold is T, the distribution is well balanced if |Y−Z|<T, and not well balanced otherwise.

If the l levels are well balanced, at 906, a number of new predictive models is set to one (i.e. N=1).

If the l levels are not well balanced, at 908, it is determined whether the l levels can be decomposed into n binary pairs (n<1). For example, the six (6) outcome possibilities from FIG. 5 can be decomposed into a single binary pair, including a first binary level of the pair of EMER-ASSIST=Yes or EMER-ASSIST=No, and a second binary level of the pair of TRANS=Yes or TRANS=No.

If the l levels can be decomposed as such, at 910, the number of new predictive models is set to n (i.e. N=n).

If the l levels cannot be decomposed as such, at 912, the l levels are grouped into L (L<1) well balanced levels, and, at 914, the number of new predictive models is set to one (i.e. N=1).

At 916, the N predictive models are trained with a training set of case notes with a known outcome.

At 918, the trained N predictive models are tested with a test set of case notes with a known outcome.

At 920, the trained and tested N predictive models are used to predict values for the missing outcomes.

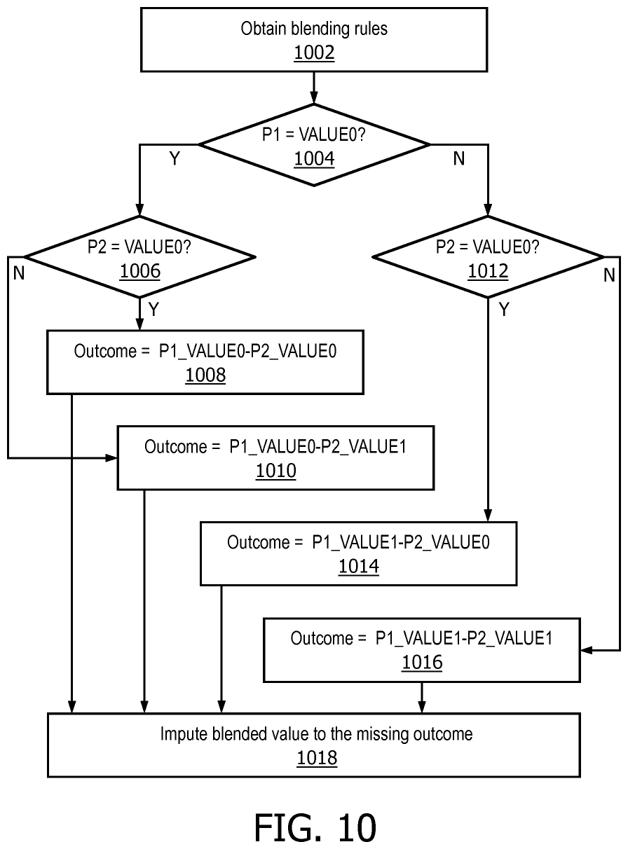

FIG. 10 illustrates an example algorithm for act 612 of FIG. 6 to blend multiple predicted values to produce a single imputed outcome. For explanatory purposes, this example is described for n=2 and two predicted values, P1 and P2.

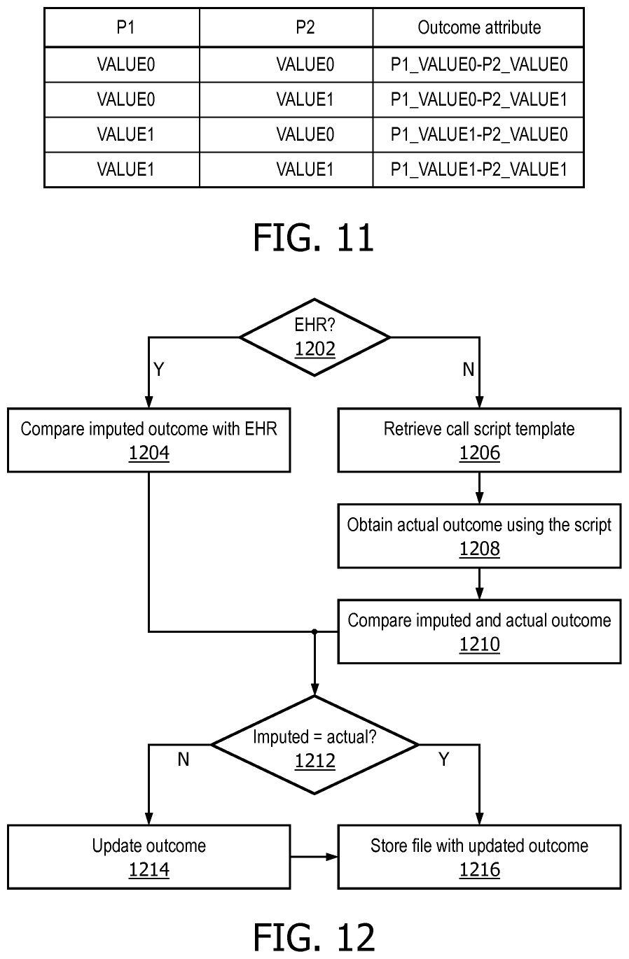

At 1002, blending rules are obtained. FIG. 11 shows an example of blending rules that will be used in this example where n=2. These blending rules show the outcome for four different pairs of the predicted values P1 and P2.

Returning to FIG. 10, at 1004, it is determined whether P1 is equal to VALUE0.

If P1 is equal to VALUE0 at 1004, at 1006, it is determined whether P2 is equal to VALUE0.

If P2 is equal to VALUE0 at 1006, at 1008, P1_VALUE0 and P2_VALUE0 are blended to create the single value P1_VALUE0-P2_VALUE0.

If P2 is not equal to VALUE0 at 1006, at 1010, P1_VALUE0 and P2_VALUE1 are blended to create the single value P1_VALUE0-P2_VALUE1.

If P1 is not equal to VALUE0 at 1004, at 1012, it is determined whether P2 is equal to VALUE0.

If P2 is equal to VALUE0 at 1012, at 1014, P1_VALUE1 and P2_VALUE0 are blended to create the single value P1_VALUE1-P2_VALUE0.

If P2 is not equal to VALUE0 at 1012, at 1016, P1_VALUE1 and P2_VALUE1 are blended to create the single value P1_VALUE1-P2_VALUE1.

At 1018, the blended value is imputed to the missing outcome.

FIG. 12 illustrates an example algorithm for act 618 of FIG. 6 to confirm imputed outcome.

At 1202, it is determined, for each user with a record with an imputed outcome, if there is an electronic health record (EHR). In one example, this can be achieved through a PERS-EHR integration interface.

If there is an EHR for a user, at 1204, the imputed outcome is compared with the outcome in the EHR. In one instance, this includes analyzing the EHR to extract emergency room (ER) and/or hospital admissions information about the outcome and comparing the extracted information with the predicted outcome to confirm the predicted outcome.

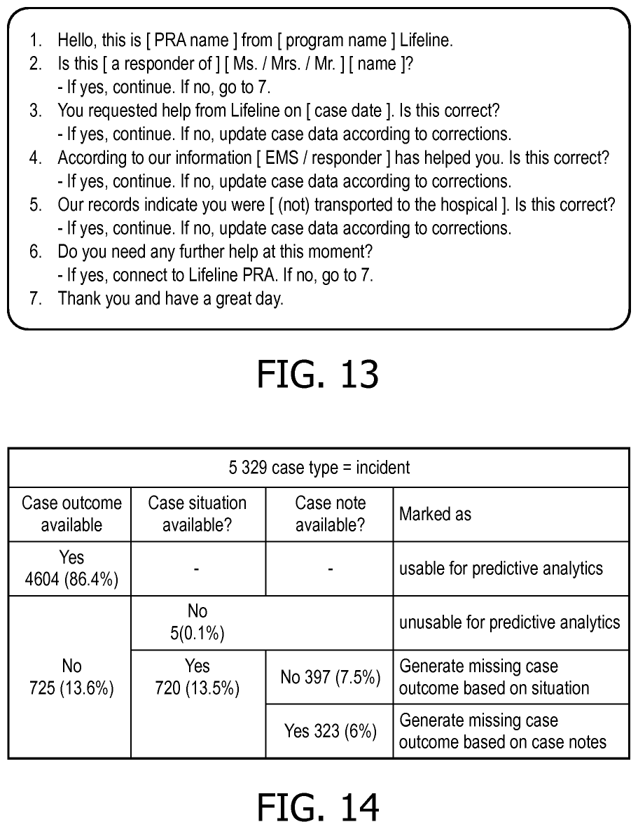

If there is no EHR for the user, at 1206, a call script template is retrieved from a set of predefined templates. The call script retrieved depends from the case type and the predicted case outcome. FIG. 13 shows an example call script.

Returning to FIG. 12, at 1208, information about the actual outcome is received using the script. For this, a call is initiated to the user and/or the responder. In one instance, this is achieved through interactive voice response (IVR) technology. For example, the call can be established through the use of voice and Dual-tone multi-frequency signaling (DTMF) tones input via keypad.

At 1210, the imputed outcome is compared with the actual information.

At 1212, it is determined if the imputed outcome is the same as the retrieved outcome.

If the predicted outcome is not the same as the actual outcome, at 1214, the record is updated with the actual outcome.

If the predicted outcome is the same as the actual outcome or after updating the imputed outcome, at 1216 the record is stored with the confirmed outcome.

A non-limiting example of using the approach described herein is described next in connection with FIGS. 14-24.

For this example, there are 5,329 records of type “incident.” The output of act 602 (FIG. 6) is shown in FIG. 14. From FIG. 14, 4,604 (86.4%) of the 5,329 type “incident” records include an outcome and are marked as “usable for predictive analytics.” The remaining 725 (13.6%) records are missing an outcome. Only 5 (0.1%) of these do not have situation/case note and are marked as “unusable for predictive analytics.”

Of the remaining 720 (13.5%), 397 (7.5%) have a situation attribute but no case note, and of 323 (6%) have a situation attribute that refers to a case note.

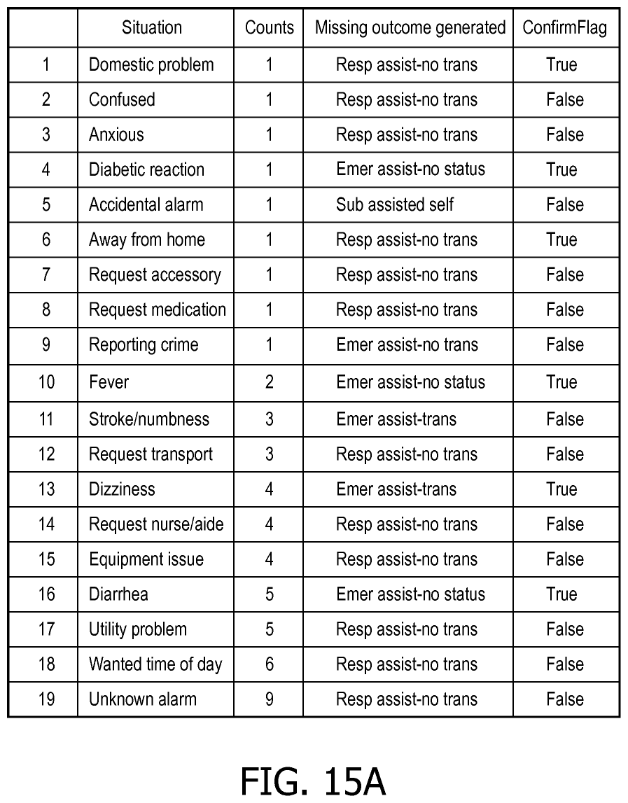

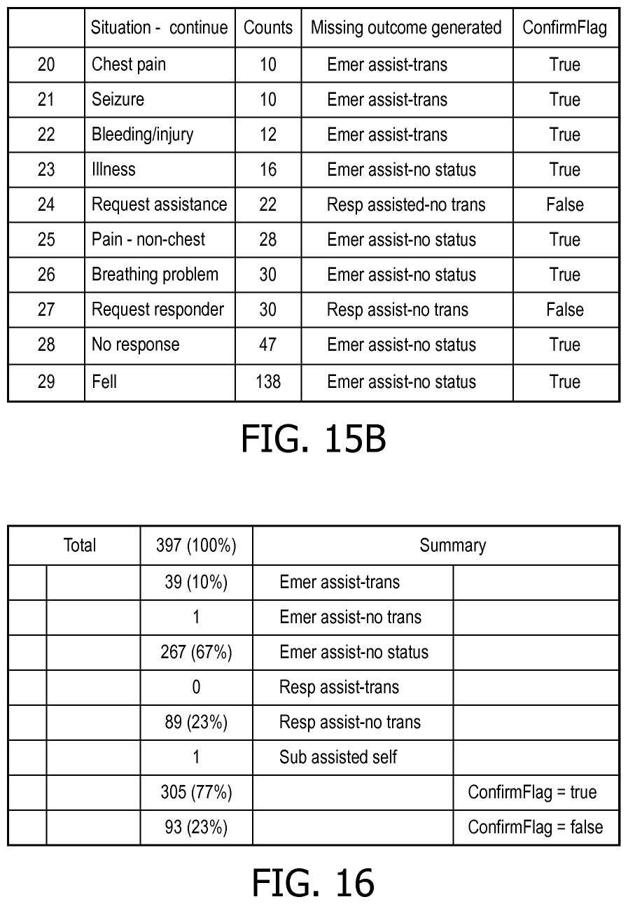

FIGS. 15A, 15B and 16 show example results a structured situation string analysis (act 606 in FIG. 6). FIG. 15A is a first part of a table and FIG. 15B is a second part of the table, which continues from the bottom of the table in FIG. 15A, as indicated by the numbering in the first column. FIG. 16 shows a summary of the information in FIGS. 15A and 15B. In this example, the situation attribute is classified into one of the six levels (l) of outcome attributes shown in FIG. 5. FIGS. 15A, 15B and 16 also indicates whether the imputed outcome is to be confirmed. The summary in FIG. 16 shows that the majority of the incident cases in this example are classified as “Emer Assist—No Status” (67%), followed by “Resp Assist—No Trans” (23%) and “Emer Assist—Trans” (10%).

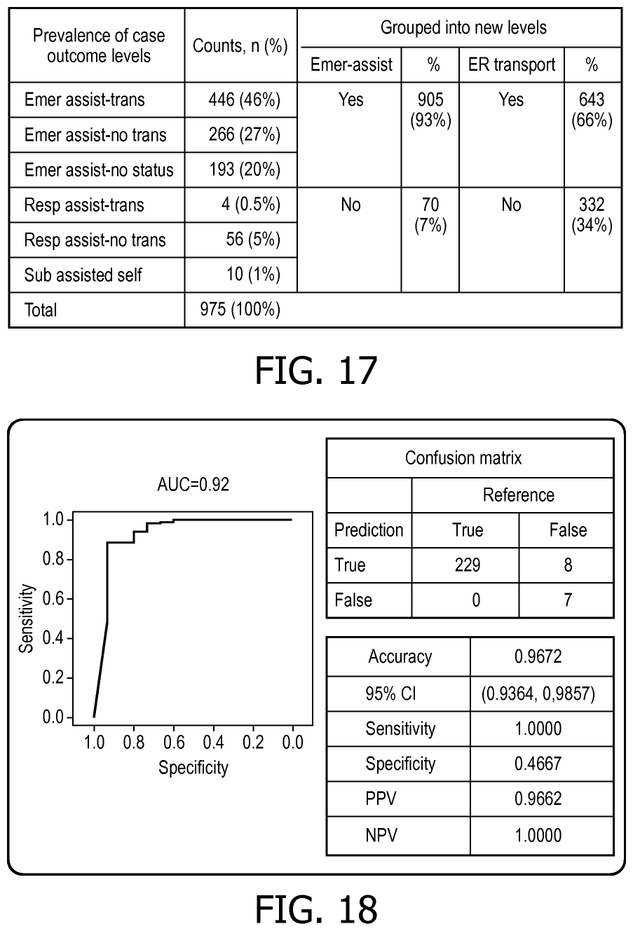

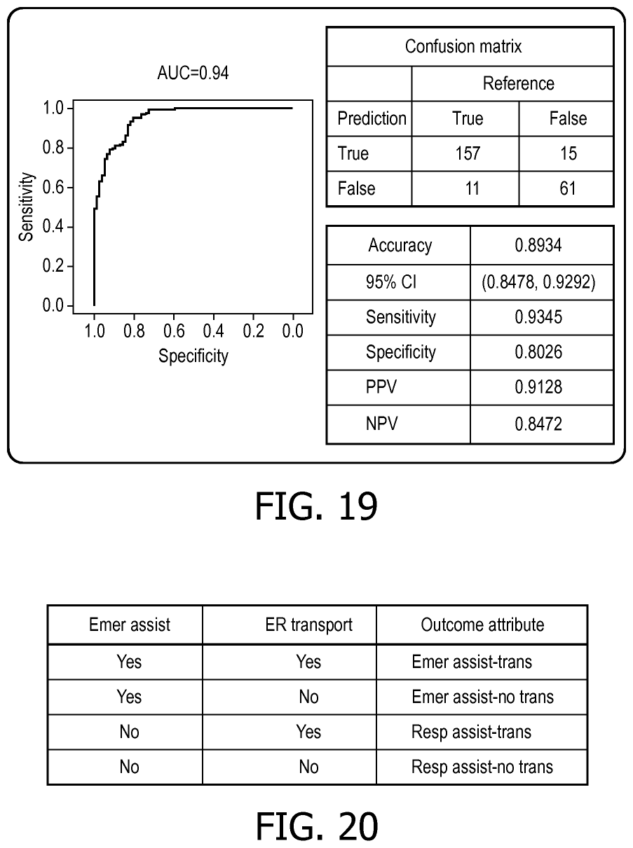

FIGS. 17, 18 and 19 show example results of a case notes analysis (act 608 in FIG. 6, in particular acts 902-912 of FIG. 9). The analysis starts with a decision how many predictive models to use to generate the missing case outcome values, which is a categorical variable with six levels (l) as shown in FIG. 5. From FIG. 17, this decision is based on the prevalence of these six levels in incidental cases with known outcomes and case notes in total 975 cases. These cases are used to build new predictive model(s). Column 2 in FIG. 17 indicates that the case outcome levels are very imbalanced as the number of occurrences range from 446 to 4. Therefore, the six levels are decomposed into two new binary parameters, Emer-Assist and ER Transport, as shown in FIG. 17. Based on this decomposition, two new models are trained and tested to predict the two new parameters. Examples of performance of the models is shown in FIGS. 18 and 19.

For this example, the blending rules shown in FIG. 11 are utilized to blend the two new parameters confirmed as described in act 612 (FIG. 6). In this example, P1=“EMER ASSIST,” P2=“ER TRANSPORT,” VALUE0=“YES,” VALUE1=“NO,” and the outcome attribute is from a group consisting of “EMER ASSIST-TRANS,” “EMER ASSIST-NO TRANS,” “RESP ASSIST-TRANS,” and “RESP ASSIST-NO TRANS.” This is shown in FIG. 20. For situation/outcome pairs that require confirmation (as indicated in FIGS. 15A and 15B), the blended outcome is confirmed as described in act 618 (FIG. 6) by either an EHR analysis or IVR calls using a script such as the script shown in FIG. 13. The record is either saved with the imputed outcome, or the outcome is updated to the correct actual outcome and the record is saved with the updated imputed outcome.

The computing system 204 (FIG. 2) can employ records with outcomes and records with imputed and/or update imputed outcomes for predictive analysis to predict users at risk of emergency transport and generate enhanced medical alerts, when needed. Additionally, using the records with the 720 imputed outcomes (which records have been ignored in prior art predictive analyses) improved the accuracy of the predicted results.

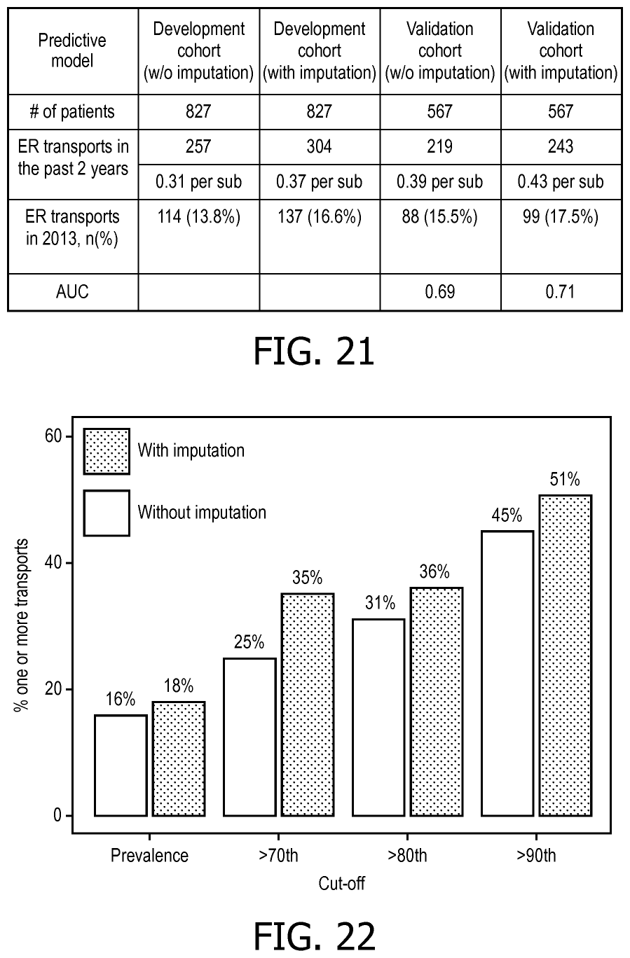

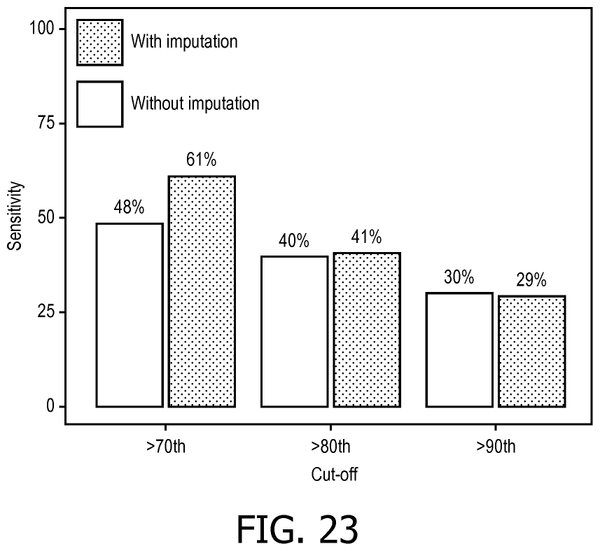

The following illustrates a non-limiting example that shows an example of a benefit of imputing an outcome attribute as described herein. For this example, a predictive model is trained/validated with historic data and outcome window, and performance is tested on a cohort without imputation and a cohort with imputation. From FIG. 21, an area under the curve (AUC) is 0.69 without imputation and 0.71 with imputation. FIG. 22 illustrates bar graphs of a positive predictive value (PPV) with imputation and without imputation. FIG. 23 illustrates bar graphs of sensitivity with imputation and without. In this example, the PPV improvement ranges up to a 5-10% absolute increase at a cutoff value, while a sensitivity was generally preserved at a same level.

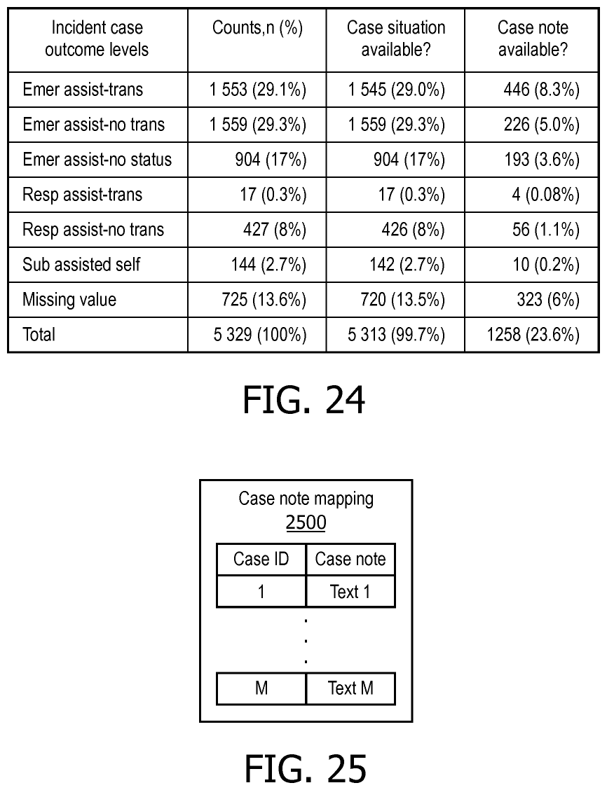

In another example, the approach described herein is applied to records with the type incident with a partly missing outcome. An example of this category is all incident cases with assigned outcome “Emer Assist—No Status”. No Status means that the call center 106 was unable to get the user's transport status upon calling back the user and/or responder. This may occur when the user or responder does not answer the call center follow-up call, the hospital did not confirm the user's admission, or the EMS dispatch center cannot provide transport information. In this example, of the 3,258 subscribers in the previous example, there are 904 (17%) incident cases with outcome “Emer Assist—No Status,” which is shown in FIG. 24.

In FIG. 24, all of them have a case situation attribute, and 193 of them also have a case note. The case situation analysis and case note analysis described herein can be used to classify them either into “Emer Assist—Trans” or Emer Assist—No Trans” category. In FIG. 24, the case situation attribute is available in almost all incident cases (5,313 (99.7%)), and 24% of all incidents have a case note available. As such, the case situation analysis and case note analysis described herein can be applied to incident cases with known outcomes to: 1) confirm the outcome which is prone to an error input by call center personnel during recording time; and 2) generate a human readable report highlighting root causes for flagging a subscriber as “high risk.”

The following describes an example to classify case outcomes based on the case notes (e.g., from a record and/or the case note database). In this example, first, separate case note fragments belonging to a single case are joined. These separate fragments result from a single case often involving different interactions with the user, even by different call centers. Case notes are then converted to lower case, and stripped from commas and dots. Words are extracted from the case notes by splitting case notes on white spaces. In one instance, the call center can use a standard list of abbreviations, e.g., “disp” means dispatch and “amb” means ambulance.

A table of the frequency of each word is generated and sorted in order of decreasing frequency. For each of the N most frequently occurring words, one-hot encoding can be used to indicate if this word was present in the case note. Thus, a large sparse matrix can be generated with one row per case and N columns for the words, where each cell is “1” if the corresponding word is present in the case note and “0” otherwise. This approach is also known as “bag-of-words”. In one instance, the number of words N can be varied from 5 to 500 to determine the optimal N required for classification of the case notes.

The set of case notes is randomly split into a training and test set using a 0.75/0.25 ratio. The training set is used to develop the case outcome classifier. The classifier(s) is developed using a machine learning technology, e.g. boosted regression trees approach referred to as extreme gradient boosting. Due to its tree-based nature, this methodology allows for automatic selection of interaction between variables. Variable importance is determined according to a ‘gain’, which is a measure for the relative contribution of the corresponding variable to the predictive model, calculated by taking the improvement in accuracy brought by a variable to the branches it is on. The boosted regression model involves tuning hyperparameters of the learning algorithm, such as the number of trees, the maximum depth of the trees (defining the degree of interaction between variables), and the learning rate. This optimization can be achieved using 5-fold cross-validation on the training set with the optimization metric determined by the AUC in the test fold.

The separate test set is used to validate the performance of the classifiers, which is depicted in FIGS. 18 and 19. The test set is also used to determine the optimal cut-off probability based on the Youden index (maximum sensitivity+specificity). Finally, the classifier is then used to impute cases with missing outcomes, by setting case outcomes to “Emergency Assistance—Transport” for those cases with a predicted probability that exceeded the cut-off. In addition to the original case data set, this new case data set with imputed case outcomes was then stored for further use in the predictive modeling of emergency hospital transport.

In another embodiment, the approach described herein is applied to pre-populate case attributes while call center personnel is in a conversation with a subscriber and types a case note. In this embodiment, the predictive models analyze the case note text as the text is entered (i.e. in real time) and predict the value of different case attributes. These values are pre-filled in the record for the personnel, who can confirm them and proceed with rounding up the case. In one instance, this will lead to improved call center efficiency and quality of recorded data.

In another embodiment, the approach described herein is applied to analyze case notes typed by a sale representative and predict the probability of finalizing an inbound call with having a new subscriber and no need for a call back.

The method(s) described herein may be implemented by way of computer readable instructions, encoded or embedded on computer readable storage medium (which excludes transitory medium), which, when executed by a computer processor(s) (e.g., CPU, microprocessor, etc.), cause the processor(s) to carry out acts described herein. Additionally, or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computing system, comprising:
a memory device configured to store missing outcome instructions; and
a processor configured to execute the missing outcome instructions, which causes the processor to:
evaluate records stored on a storage device of a call center of a system,
wherein the storage device is configured to store electronic records of calls from a user site to the call center, and each record includes at least a situation attribute field and an outcome attribute field;
identify records of the stored records which include, at least one of, a value in the situation attribute or case note text in a case note database, but no value in the outcome attribute;
impute an outcome to the outcome attribute field of each identified record of the stored electronic records using at least one of the situation attributes and the case note text to predict the missing outcome, and
store the identified electronic records that include the imputed outcomes in the storage device.

2. The system of claim 1, wherein the situation attribute is from a group consisting of a structured situation string and a reference to a case note, and the processor is further configured to identify whether the situation attribute is the structured situation string or the reference to the case note.

3. The system of claim 2, in response to identifying the situation attribute as the structured situation string and no case note available, the processor is further configured to determine the outcome to impute to the record using a look up table which maps structured situation strings to imputed outcomes.

4. The system of claim 2, in response to at least one of identifying the situation attribute as the reference to the case note and identifying the database has the case note text, the processor is further configured to determine a distribution of a first set of outcome attributes of reference records with both an outcome and a case note, and determine whether the distribution is balanced across outcome attributes based on a predetermined threshold level.

5. The system of claim 4, in response to determining the distribution is balanced, the processor creates a set of only one new predictive model for the balanced distribution of outcome attributes.

6. The system of claim 4, in response to determining the distribution is not balanced and the first set can be decomposed into a second smaller set of outcome attributes, the processor decomposes the outcomes of the first set into the second smaller set and creates a set of multiple new predictive models, one for each of the outcomes in the second smaller set.

7. The system of claim 4, in response to determining the distribution is not balanced and the first set cannot be decomposed into a second smaller set of outcome attributes, the processor groups the outcome attributes into a third set of balanced outcome attributes, which is smaller than the first set, and creates a set of only one new predictive model groups.

8. The system of claim 5, wherein the processor is further configured to train the set of new predictive models with a training set of case notes with a known outcome.

9. The system of claim 8, wherein the processor is further configured to test the trained set of new predictive models with a testing set of case notes with a known outcome.

10. The system of claim 9, wherein the processor is further configured to employ the trained and tested set of new predictive models to impute outcome attributes for each record with the situation attribute or a case note but no outcome attribute.

11. The system of claim 10 wherein the processor is further configured to blend the outcome attributes parts into a single outcome attribute in response to the set of new predictive models including multiple outcome attributes.

12. The system of claim 10, wherein the processor is further configured to confirm at least one of the imputed outcomes.

13. The system of claim 1, wherein the memory device is further configured to store predictive models, including the trained and tested set of predictive models, and the processor is further configured to execute the stored predictive models to predict, for a user of the system, a risk of emergency transport based on the predictive models.

14. The system of claim 13, wherein the processor is further configured to generate and transmit a notification to a clinical site in response to the predicted outcome indicating a user is at risk of transportation to a healthcare facility within a specified time period.

15. A method, comprising:
identifying stored records, which include at least a situation attribute field and an outcome attribute field, that have at least one of a situation attribute and case note text in a case note database, and no outcome attribute;
determining whether the situation attribute field includes a structured situation string or a reference to a case note in response to the situation attribute field including a situation attribute;
performing a structured situation string analysis to impute an outcome to one or more of the records missing an outcome attribute in response to the situation attribute field including the structured situation string;
performing a case note analysis to impute an outcome to one or more of the records missing an outcome attribute in response to at least one of the situation attribute field including the reference to the case note and the case note database including the case note text;
employing records with outcome attributes and imputed outcome attributes to generate predictive models;
employing the predictive models to predict a health state of a user;
imputing the predicted health state of the user to a record of the identified records; and
storing the record with the imputed predicted health state of the user.

16. (canceled)

17. (canceled)

18. (canceled)

19. (canceled)

20. (canceled)
